(19) United States
(12) Patent Application Publication
(10) Pub. No.: US 2024/0352441 A1
(43) Pub. Date: Oct. 24, 2024

(54) METHOD FOR REDUCING ENDOTOXIN LEVELS IN NUCLEIC ACID PURIFICATION

(71) Applicant: MERCK PATENT GMBH, DARMSTADT (DE)

(72) Inventors: Anja HEINEN-KREUZIG, Darmstadt (DE); Andre KIESEWETTER, Darmstadt (DE); Andreas STEIN, Darmstadt (DE); Akshat GUPTA, Burlington, MA (US)

(73) Assignee: MERCK PATENT GMBH, DARMSTADT (DE)

(21) Appl. No.: 18/294,123

(22) PCT Filed: Aug. 3, 2022
(86) PCT No.: PCT/EP2022/071786

§ 371 Date: Jan. 31, 2024

Related U.S. Application Data
(60) Provisional application No. 63/229,666, filed on Aug. 5, 2021.

(51) Int. Cl. C12N 15/10; B01D 15/12; B01D 15/36; B01D 15/42

(52) U.S. Cl. CPC C12N 15/101; B01D 15/12; B01D 15/363; B01D 15/424

(57) ABSTRACT

The present invention relates to a method for reducing endotoxin levels or removing endotoxins from nucleic acids. For this a non-ionic detergent selected from the group of alkylglycosides and secondary alcohol alkoxylates or mixtures thereof is added prior or during anion exchange chromatographic purification of the nucleic acids using a membrane or monolith-based sorbent.

The present invention relates to a method for reducing endotoxin levels or removing endotoxins from nucleic acids. For this a non-ionic detergent is added during anion exchange chromatographic purification of the nucleic acids using a membrane or monolith-based sorbent.

The demand for rapid and efficient methods for obtaining high-purity nucleic acids like plasmid DNA from biological sources is constantly increasing owing to the increasing importance of recombinant DNA for exogenous expression or therapeutic applications. In particular, the demand for purification methods which can also be carried out on a larger scale is also increasing. The use of highly pure plasmid DNA is crucial in various applications like polymerase chain reaction (PCR) amplification, DNA sequencing, in vitro mRNA synthesis, and subcloning of transgenes. Therefore, protocols for generating plasmid DNA with high yield and quality have earned serious attention.

Many known methods for the purification of, in particular, relatively large amounts of nucleic acids like plasmid DNA include a chromatographic purification step. The efficiency of this step generally also determines the efficiency and effectiveness of the manufacturing process.

A further problem in the purification of especially plasmid DNA is caused by the impurities from which the plasmid DNA is to be separated. These are firstly genomic DNA and RNA. Another impurity when purifying nucleic acids are endotoxins. Endotoxins are lipopolysaccharides (LPSs) which are located on the outer membrane of Gram-negative host cells, such as, for example, Escherichia coli. During lysis of the cells, LPSs and other membrane constituents are released in addition to the plasmid DNA. Endotoxins can be present in cells in a number of approximately 3.5×10⁶ copies per cell (Escherichia coli and Salmonella Typhimurium Cell. and Mol. Biology, J. L. Ingraham et al. Eds., 1987, ASM) and thus exceed the number of plasmid DNA molecules by a factor of more than 10⁴. For this reason, plasmid DNA obtained from Gram-negative host cells often contains large amounts of endotoxins. However, these substances result in a number of undesired side reactions (Morrison and Ryan, 1987, Ann. Rev. Med. 38, 417-432; Boyle et al. 1998, DNA and Cell Biology, 17, 343-348). If it is intended to employ the plasmid DNA for e.g. gene therapy, it is of extreme importance that, for example, inflammatory or necrotic side reactions due to the impurities do not occur. There is therefore a great demand for effective methods for reducing endotoxin concentrations to the lowest possible levels.

Known methods for reducing endotoxin levels are based on a plurality of purification steps, frequently using anion-exchange chromatography. Firstly, the host cells are digested by known methods, such as, for example, alkaline lysis. Other lysis methods, such as, for example, the use of high pressure, boiling lysis, the use of detergents or digestion by lysozyme, are also suitable.

The plasmid DNA in the medium obtained in this way, a “clarified lysate”, is principally contaminated by relatively small cell constituents, chemicals from the preceding treatment steps, RNA, proteins and endotoxins. The removal of these impurities usually requires a plurality of subsequent purification steps, anion-exchange chromatography being one possibility.

A disadvantage of anion-exchange chromatography is that a considerable amount of endotoxins is bound together with the plasmid DNA and cannot be sufficiently separated in this way. In order to reduce the endotoxin levels, further purification steps, such as, for example, chromatographic steps (gel filtration) or precipitation with isopropanol, ammonium acetate or polyethylene glycol, are therefore necessary. Purification methods which combine chromatographic methods, such as, for example, anion-exchange chromatography, and additional endotoxin removal steps enable plasmid DNA having an endotoxin content of less than 50 EU/mg of plasmid DNA to be obtained. However, methods of this type are usually complex, time-consuming and of only limited suitability for the purification of relatively large amounts of DNA.

WO 95/21179 describes a method for the reduction of endotoxin levels in which a clarified lysate is firstly pre-incubated with an aqueous salt solution and detergents. This is followed by purification by ion-exchange chromatography, in which the ion-exchange material is washed with a further salt solution, and the plasmid DNA is eluted and subsequently purified further, for example by isopropanol precipitation. This method likewise has the above-mentioned disadvantages.

U.S. Pat. No. 6,617,443 discloses a method for removing endotoxins from nucleic acid preparations using a salt-free detergent solution and sorbents whose functional groups are bonded to tentacles.

WO2009/129524 discloses a method for purifying plasmid DNA comprising contacting the plasmid DNA with a zwitterionic detergent.

U.S. Pat. No. 6,428,703 describes a method for purifying biological macromolecules by contacting them with a non-ionic detergent and performing a chromatographic purification.

All of these documents show ways of purifying plasmid DNA from endotoxins. But there is nevertheless a need for a process combining enhanced performance with a high effectivity.

Downstream processes in the biopharmaceutical and biotechnological industries usually rely on chromatographic steps with bead-based resins in a packed-bed column as the stationary phase. The resins typically have diameters between 30 and 500 μm and generally provide an efficient chromatographic technique with high binding capacity. However, the method is rather slow and represents a major cost in biomolecules production, as the transport of solute molecules to the binding sites inside resin pores is limited by intra-particle diffusion. The pressure drop over the column is high even at low flow rates and increases during processing due to bed consolidation and column blinding. Consequently, several other innovative stationary phases, including monoliths and membranes, have been developed in the last few decades as possible alternatives to classical chromatographic supports. The main advantage of using membranes or monoliths is attributed to short diffusion times, as the interactions between molecules and active sites in the membrane or monolith occur in convective through-pores rather than in stagnant fluid inside the resin pores. Therefore, membrane and monolith chromatography has the potential to operate at high flow rates and low pressure drops.

But as described above membrane or monolith-based chromatography, among others due to the absence of pore diffusion and the higher flow rates, might show different chromatographic behavior and thus different separation properties.

It has been found that the high performance when using a membrane or monolith as chromatographic matrix can be combined with high efficiency by performing plasmid DNA purification using an anion exchange membrane or monolith in combination with certain types of non-ionic detergents. It was further found that using the process of the invention subsequent determination of residual endotoxin can be performed without interference of the detergents.

The present invention is therefore directed to a method for depletion or removal of endotoxins from nucleic acids comprising
a) Providing a sample comprising said nucleic acids and endotoxins
b) Subjecting the sample of step a) to a chromatographic separation on a membrane or monolith comprising anion exchange groups whereby the sample is contacted with a non-ionic detergent selected from the group of alkylglycosides and secondary alcohol alkoxylates or mixtures thereof.

In a preferred embodiment step b) comprises
i) Loading the sample comprising said nucleic acids and endotoxins onto the membrane or monolith comprising anion exchange groups
ii) Washing the membrane or monolith with a wash buffer
iii) Eluting the nucleic acids bound to the membrane or monolith with an elution buffer

In one embodiment the sample is contacted with the non-ionic detergent prior to step b).

In another embodiment the nucleic acids are contacted with the non-ionic detergent by washing the membrane or monolith in step ii) with a wash buffer comprising a non-ionic detergent.

Preferably the detergent is added to the sample and/or to the wash buffer such that it has a concentration therein ranging from 0.01% to 10% (w/v).

In a preferred embodiment the non-ionic detergent is an alkylglycoside. In a very preferred embodiment, it is a C8-16 alkyl glycoside.

In a preferred embodiment the nucleic acids comprise or consist of plasmid DNA.

In a preferred embodiment the nucleic acids are contacted with a solution comprising 0.01 to 10% (w/v) of the non-ionic detergent.

In a preferred embodiment the membrane is a hydrogel membrane.

In a preferred embodiment step ii) comprises two or more wash steps whereby one wash step is done with a wash buffer comprising ethanol. In one embodiment the process of the invention provides for nucleic acids which are depleted from endotoxins more effectively as with the otherwise same process but using Triton® X100 as the only detergent.

In one embodiment the process further comprises a step c) detecting residual endotoxin in the nucleic acids resulting from step b).

In a preferred embodiment the detection in step c) is done by LAL assay or recombinant factor based assays, especially by LAL assay.

In a preferred embodiment the detection in step c) is done directly in the eluate of the chromatographic separation, without any further treatment of the eluate.

### Definitions

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a ligand” includes a plurality of ligands and reference to “an antibody” includes a plurality of antibodies and the like.

Unless defined otherwise, all technical and scientific terms used herein have the same meaning as commonly understood by one of ordinary skill in the art to which this invention is related. The following terms are defined for purposes of the invention as described herein.

Nucleic acids that may be purified according to the method of the present invention, also called target nucleic acids, by depletion or removal of endotoxins include DNA, RNA and chimeric DNA/RNA molecules, and may be from any biological source including eukaryotic and prokaryotic cells, or may be synthetic. Nucleic acids that may be purified include chromosomal DNA fragments, ribosomal RNA, mRNA, snRNAs, tRNA, plasmid DNA, viral RNA or DNA, synthetic oligonucleotides, ribozymes, and the like. Of particular interest are plasmid DNAs encoding therapeutic genes. By “therapeutic genes” is intended to include functional genes or gene fragments which can be expressed in a suitable host cell to complement a defective or under-expressed gene in the host cell, as well as genes or gene fragments that, when expressed, inhibit or suppress the function of a gene in the host cell including, e.g., antisense sequences, ribozymes, transdominant inhibitors, and the like.

Thus, e.g., viral DNA or RNA may be purified from prokaryotic or eukaryotic viruses, in which the viral particles are initially purified from cultures or cells permissive for viral infection in accordance with conventional techniques, e.g., from bacterial, insect, yeast, plant or mammalian cell cultures.

The term “plasmid DNA” refers to any distinct cell-derived nucleic acid entity that is not part of or a fragment of the host cell's primary genome. As used herein, the term “plasmid” may refer to either circular or linear molecules composed of DNA or DNA derivatives. The term “plasmid DNA” may refer to either single stranded or double stranded molecules. Plasmid DNA includes naturally occurring plasmids as well as recombinant plasmids encoding a gene of interest including, e.g., marker genes or therapeutic genes.

Plasmids are typically epigenomic circular DNA molecules having a length of between 4 and 20 kB, which corresponds to a molecular weight of between 2.6×10 6 and 13.2×10 6 Daltons often capable of autonomous replication in a producing cell. Even in their compact form (super coil), plasmid DNA molecules normally have a size of several hundred nm.

As used herein, and unless stated otherwise, the term “sample” refers to any composition or mixture that contains nucleic acids. Samples may be derived from biological or other sources. Biological sources include eukaryotic and prokaryotic sources, such as plant and animal cells, tissues and organs. The sample may also include diluents, buffers, detergents, and contaminating species, debris and the like that are found mixed with the target molecule. The sample may be “partially purified” (i.e., having been subjected to one or more purification steps, such as filtration steps) or may be obtained directly from a host cell or organism producing the nucleic acid (e.g., the sample may comprise harvested cell culture fluid).

The term “impurity” or “contaminant” as used herein, refers to any foreign or objectionable molecule, including one or more host cell proteins, endotoxins, lipids and one or more additives which may be present in a sample containing the nucleic acids that is being separated from one or more of the foreign or objectionable molecules using a process of the present invention. One contaminant that is depleted or removed with the process of the present invention are endotoxins.

The terms “purifying,” “separating,” or “isolating,” as used interchangeably herein, refer to increasing the degree of purity of the target nucleic acids from a composition or sample comprising the target nucleic acids and one or more impurities. Typically, the degree of purity of the target nucleic acid is increased by removing (completely or partially) at least endotoxins from the composition.

The term “chromatography” refers to any kind of technique which separates an analyte of interest (e.g. a target nucleic acid) from other molecules present in a sample. Usually, the target nucleic acid is separated from other molecules as a result of differences in rates at which the individual molecules of the mixture bind to and/or migrate through a chromatography matrix under the influence of a moving phase.

The term “matrix” or “chromatography matrix” are used interchangeably herein and refers to a solid phase though which the sample migrates in the course of a chromatographic separation. The matrix typically comprises a base material and ligands covalently bound to the base material. The matrix of the present invention comprises or consists of a membrane or monolith, preferably the base material is a membrane or monolith, most preferred a membrane.

A “ligand” is a functional group that is part of the chromatography matrix, typically it is attached to the base material of the matrix, and that determines the binding properties and interaction properties of the matrix. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). It is also possible that one ligand has more than one binding/interaction property. The matrix of the present invention comprises at least anion exchange groups. These might for example be strong anion exchange groups, such as trimethylammonium chloride or weak anion exchange groups, such as N,N diethylamino or DEAE. The matrix may additionally comprise further other types of ligands so that the matrix is a mixed mode matrix. Such ligands may e.g. have hydrophobic interaction groups, such as phenyl, butyl, propyl, hexyl.

The ligands can be attached to the base material of the matrix by any type of covalent attachment. Covalent attachment can for example be performed by directly bonding the functional groups to suitable residues on the base material like OH, NH₂, carboxyl, phenol, anhydride, aldehyde, epoxide or thiol etc. It is also possible to attach the ligands via suitable linkers. It is also possible to generate the matrix by polymerizing monomers comprising the ligands and a polymerizable moiety. Examples of matrices generated by polymerization of suitable monomers are polystyrene, polymethacrylamide or polyacrylamide based matrices generated by polymerizing suitable styrole or acryloyl monomers.

In another embodiment the stationary phase can be generated by grafting the ligands onto the base material or from the base material. For grafting from processes with controlled free-radical polymerisation, such as, for example, the method of atom-transfer free-radical polymerisation (ATRP), are suitable. A very preferred one-step grafting from polymerisation reaction of acrylamides, methacrylates, acrylates, methacrylates etc. which are functionalized e.g. with ionic, hydrophilic or hydrophobic groups can be initiated by cerium(IV) on a hydroxyl-containing support, without the support having to be activated.

When the chromatography matrix is used in a chromatographic separation it is typically used in a separation device, also called housing, as a means for holding the matrix.

In one embodiment, the device comprises a housing with an inlet and an outlet and a fluid path between the inlet and the outlet. In a preferred embodiment the device is a chromatography column. Chromatography columns are known to a person skilled in the art. They typically comprise cylindrical tubes or cartridges filled with the stationary phase as well as filters and/or means for fixing the stationary phase in the tube or cartridge and optionally connections for solvent delivery to and from the tube or cartridge. The size of the chromatography column varies depending on the application, e.g. analytical or preparative. In one embodiment the column or generally the separation device is a single use device.

The term “anion exchange matrix” is thus used herein to refer to a chromatography matrix which carries at least anion exchange groups. That means it typically has one or more types of ligands that are positively charged under the chromatographic conditions used, such as quaternary amino groups.

A “buffer” is a solution that resists changes in pH by the action of its acid-base conjugate components. Various buffers which can be employed depending, for example, on the desired pH of the buffer are described in Buffers. A Guide for the Preparation and Use of Buffers in Biological Systems, Gueffroy, D., ed. Calbiochem Corporation (1975). Non-limiting examples of buffers include MES, MOPS, MOPSO, Tris, HEPES, phosphate, acetate, citrate, succinate, and ammonium buffers, as well as combinations of these.

According to the present invention the term “buffer” or “solvent” is used for any liquid composition that is used to load, wash, elute, re-equilibrate, strip and/or sanitize the chromatography matrix.

When “loading” a chromatography column in bind and elute mode, the sample or composition comprising the target molecule and one or more impurities is loaded onto a chromatography column. In preparative chromatography, the sample is preferably loaded directly without the addition of a loading buffer. If a loading buffer is used, the buffer has a composition, a conductivity and/or pH such that the target nucleic acid is bound to the stationary phase while ideally all the impurities like the endotoxins are not bound and flow through the column. Typically, the loading buffer, if used, has the same or similar composition as the equilibration buffer used to prepare the column for loading.

The final composition of the sample loaded on the column is called feed. The feed may comprise the sample and the loading buffer but preferably it is only the sample.

By “wash” or “washing” a chromatography matrix is meant passing an appropriate liquid, e.g. a buffer through or over the matrix. Typically washing is used to remove weakly bound contaminants from the matrix in bind/elute mode prior to eluting the target molecule. Additionally, wash steps can be used to reduce levels of residual detergents, enhance viral clearance and/or alter the conductivity carryover during elution.

To “elute” a molecule (e.g. the target nucleic acid) from a matrix means that the molecule is removed therefrom. Elution may take place by altering the solution conditions such that a buffer different from the loading and/or washing buffer competes with the molecule of interest for the ligand sites on the matrix or alters the equilibrium of the target molecule between stationary and mobile phase such that it favors that the target molecule is preferentially present in elution buffer.

A non-limiting example is to elute a molecule from an ion exchange resin by altering the ionic strength of the buffer surrounding the ion exchange material such that the buffer competes with the molecule for the charged sites on the ion exchange material.

A membrane as chromatographic matrix can be distinguished from particle-based chromatography by the fact that the interaction between a solute, e.g. the target nucleic acids or contaminants, and the matrix does not take place in the dead-ended pores of a particle, but mainly in the through pores of the membrane. Exemplary types of membranes are flat sheet systems, stacks of membranes, microporous polymer sheets with incorporated cellulose, polystyrene or silica-based membranes as well as radial flow cartridges, hollow fiber modules and hydrogel membranes.

Preferred are hydrogel membranes. Such membranes comprise a membrane support and a hydrogel formed within the pores of said support. The membrane support provides mechanical strength to the hydrogel. The hydrogel determines the properties of the final product, like pore size and binding chemistry.

The membrane support can consist of any porous membrane like polymeric membranes, ceramic based membranes and woven or non-woven fibrous material. Suitable polymeric materials for membrane supports are cellulose or cellulose derivatives as well as other preferably inert polymers like polyethylene, polypropylene, polybutylenterephthalate or polyvinylidene-difluoride.

The hydrogels can be formed through in-situ reaction of one or more polymerizable monomers with one or more crosslinkers and/or one or more cross-linkable polymers to form a cross-linked gel that has preferably macropores. Suitable polymerizable monomers include monomers containing vinyl or acryl groups. Preferred are monomers comprising an additional functional group that either directly forms the ligand of the matrix or is suitable for attaching the ligands. Suitable crosslinkers are compounds containing at least two vinyl or acryl groups. Further details about suitable membrane supports, monomers, crosslinkers etc. as well as suitable production conditions can be found in WO04073843 and WO2010/027955. Especially preferred are membranes made of an inert, flexible fiber web support comprising assembly within and around the fiber web support a porous polyacrylamide hydrogel with quaternary ammonium groups (strong anion exchange groups), like Natrix® Q Chromatography membrane, Merck KGaA, Germany.

Depending on the membrane device used, the respective processes are conducted by different operating principles like dead-end operation, cross-flow operation and radial flow operation systems. Dead-end operation is preferred.

Examples of suitable membranes to be used in the method of the present invention are
Membranes with a polyethersulfone (PES)-based support and a cross-linked polymeric coating, functionalized with quaternary ammonium groups (strong anion exchange groups), like Mustang® Q, Pall.
Membranes made of stabilized reinforced cellulose, functionalized with quaternary ammonium groups (strong anion exchange groups) or with DEAE groups (diethylaminoethyl, weak ion exchange groups), like Sartobind® membranes, Sartorius.
Membranes made of stabilized reinforced cellulose, comprising a hydrogel with quaternary ammonium groups (strong anion exchange groups), like Sartobind® Jumbo Membranes made of stabilized reinforced cellulose, functionalized with quaternary ammonium groups (strong anion exchange groups), like Sartobind® Jumbo membranes, Sartorius.
Membranes made of a fine fiber non-woven scaffold comprising a hydrogel with quaternary ammonium groups (strong anion exchange groups), like 3M™ Emphaze™ AEX Hybrid Purifier, 3M.
Membranes made of an inert, flexible fiber web support comprising within and around the fiber web support a porous polyacrylamide hydrogel with quaternary ammonium groups (strong anion exchange groups), like Natrix® Q Chromatography membrane, Merck KGaA, Germany.

A monolith or a monolithic sorbent, similar to a membrane, has through pores, like interconnected channels, so that liquid can flow from one side of the monolith, through the monolith, to the other side of the monolith.

Since the mobile phase is flowing through these through pores, molecules to be separated are transported by convection rather than by diffusion. Due to their structure monolithic sorbents show flow rate independent separation efficiency and dynamic capacity.

The monolith is typically formed in situ from reactant solutions and can have any shape or confined geometry, typically with frit-free construction, which guarantees convenience of operation. Preferably, monolithic materials have a binary porous structure, mesopores and macropores. The micron-sized macropores are the through pores and ensure fast dynamic transport and low backpressure in applications; mesopores contribute to sufficient surface area and thus high loading capacity.

The monoliths can be made of organic, inorganic or organic/inorganic hybrid materials. Preferred are organic polymer based monoliths.

The synthesis of organic polymer monoliths is typically done by a one-step polymerization providing a tunable porous structure with tailored functional groups. Generally, a pre-polymerization mixture consisting of the monomers, crosslinkers, porogenic solvents, and initiators in an appropriate ratio is polymerized in a suitable container, also called mould, determining the format of the monolith. Polymerization is typically initiated by heating, use of UV radiation, microwave or γ-ray radiation in the presence of initiators. After reaction for the prescribed time at an appropriate temperature, the resulting material is typically washed with solvents to remove unreacted components and porogenic solvents. Suitable organic polymers are polymethacrylates, polyacrylamides, polystyrenes, polyurethanes, etc., like Poly(methacrylic acid-ethylene dimethacrylate), Poly(glycidyl methacrylate-ethylene dimethacrylate) or Poly(acrylamide-vinylpyridine-N,N′-methylene bisacrylamide).

Inorganic monoliths can be made of silica or other inorganic oxides. Preferably they are made of silica. Silica monoliths are normally prepared via a sol-gel method with phase separation. This mainly includes hydrolysis, condensation, and polycondensation of silica precursors. Typically, tetraethoxysilane (TEOS) or tetramethylorthosilicate (TMOS) is distributed in a suitable solvent in the presence of a porogen (e.g. poly(ethylene glycol) (PEG)), followed by the addition of a catalyst, acid or base, or a binary catalyst, acid and base in sequence. After reaction for a prescribed time, the resulting gel-like product is washed with solvents to remove unreacted precursor, porogen, and catalyst, followed by the proper post treatment, typically a heat treatment.

The monoliths can be modified with suitable functional groups, preferably at least ion exchange groups, to generate the targeted interaction with the sample comprising the target molecule and thus the targeted separation.

Typically the monoliths are contained in a housing like a column.

Alkyl glycosides, also called alkyl polyglycosides, comprise a saccharide and an alkyl chain linked to the saccharide, typically via the anomeric carbon. The saccharide can be a monosaccharide like glucose or a di- or oligo-saccharide like maltose. Regardless of the type of the saccharide unit, the molecules are simply called glycosides. Preferably, the saccharide is glucose. The alkyl chain is preferably a straight, saturated alkyl chain having 8 to 16 C-atoms. An alkyl glycoside to be used in the method of the present invention can also be a mixture of two or more different alkyl glycosides having different saccharide moieties and/or alkyl chains with different chain lengths. Preferred are alkyl glucosides with an alkyl chain length between 8 and 10 C-atoms. Especially preferred is Triton® CG-110, Merck KGaA, Germany.

Secondary alcohol alkoxylates contain an ethylene and/or a propylene oxide chain attached to a secondary alcohol. The secondary alcohol preferably has 8 to 18 carbons and the ethylene/propylene oxide chain preferably has 3 to 12 ethylene oxide and/or propylene oxide units. A secondary alcohol alkoxylate can also be a mixture of different secondary alcohol alkoxylates having different alcohol chains and/or different numbers of ethylene oxide units and/or propylene oxide units. Preferred secondary alcohol alkoxylates to be used in the method of the present invention are 2-ethyl hexanol ethylene oxide-propylene oxide copolymers according to Formula I.

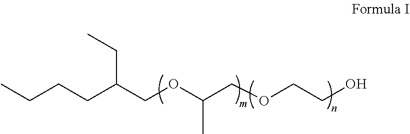

whereby m and n are a number between 1 and 11 and m+n is 3 to 12. Such secondary alcohol alkoxylates are commercially available as Ecosurf® EH, Merck KGaA, Germany, or Dow Inc.

Especially preferred is Ecosurf® EH-9.

Other preferred secondary alcohol alkoxylates to be used in the method of the present invention are secondary alcohol ethoxylates made from secondary alcohols with 11 to 15 carbons and carrying 3 to 12 ethylene oxide units. An especially preferred group of such secondary alcohol ethoxylates is shown in Formula II comprising 9 ethylene oxide units.

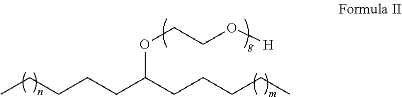

Such compounds are commercially available as Tergitol® 15-S-9, Merck KGaA, Germany.

### DETAILED DESCRIPTION

The nucleic acids to be purified according to the method of the present invention may originate from any natural, genetic-engineering or biotechnological source, such as, for example, prokaryotic cell cultures. If nucleic acids from cell preparations are to be purified, the cells are firstly digested by known methods, such as, for example, lysis. If the sample to be purified has already been pre-treated in another way, lytic digestion is unnecessary. For example, the sample can be obtained from biological material by removal of the cell debris and a precipitate of RNA, from nucleic acid samples which have already been pre-purified and, for example, are present in buffer, or alternatively from nucleic acid solutions which have been formed after amplification and still contain endotoxin impurities. Filtration, precipitation or centrifugation steps may be necessary. The person skilled in the art is able to select a suitable digestion method depending on the source of the nucleic acids to be purified. In any case, the sample to be purified should, for the method according to the invention, be present in a medium which does not form precipitates or cause other undesired side reactions on addition of a detergent solution. The sample is preferably a lysate obtained from cells, such as, for example, a clarified lysate.

For the purification of plasmid DNA from E. coli, the cells are, for example, firstly lysed by alkaline lysis with NaOH/SDS solution. Addition of an acidic potassium-containing neutralization buffer then causes the formation of a precipitate, which can be removed by centrifugation or filtration. The clear supernatant remaining, the clarified lysate, can be employed as starting material, i.e. as sample, for the method according to the invention. It is also possible firstly to concentrate or pre-purify the clarified lysate by known methods, such as dialysis or precipitation.

The sample comprising the nucleic acids and the endotoxins and potentially other impurities from which the nucleic acids shall be purified is then subjected to a chromatographic separation on a membrane or monolith-based chromatography matrix comprising anion exchange groups. For this the sample is loaded onto the chromatography matrix. The final composition of the sample loaded onto the matrix is called the feed. In one embodiment of the present invention the feed does not comprise any detergent. In another embodiment the feed comprises a non-ionic detergent selected from the group of alkylglycosides and secondary alcohol alkoxylates or mixtures thereof. Typically the concentration of the non-ionic detergent in the feed, if present, is between 0.01% and 10% (w/v), preferably between 0.1% and 1.5% (w/v). The non-ionic detergent can be added to the sample directly before loading onto the column by in-line mixing or it can be, preferably, added to the sample in batch prior to loading. For this the sample is preferably mixed with the detergent until the detergent is dissolved. Mixing can for example be performed for a time between 5 and 60 minutes. Typically, the feed preparation and also the chromatographic separation are performed at or around room temperature. But it is also possible to work at temperatures between 5 and 35° C.

The feed preferably is adjusted to an electrolytic conductivity between 40 to 90 mS/cm, most preferably to 75 and 85 mS/cm.

Conductivity adjustment is done by addition of salt, salt concentrate solutions, or, respectively, dilution with a low conductivity buffer or neat water. For feed conductivity adjustment by salt supplementation preferably sodium or potassium chloride are used, but any other salt commonly used in purification applications such as e.g. salts from sulfate, acetate, carbonate/bicarbonate, phosphate or citrate might be considered as well.

The feed typically shows pH values between 4.5 to 5.5 but the method might also be conducted to feeds showing pH values ranging from 4.0 up to 9.0.

Column equilibration and wash buffers are typically buffers matching the pH and conductivity of feed loaded onto the chromatography material. Typically buffers with pH below 6.0 and conductivity between 40 to 90 mS/cm are selected but buffers out of that range are applicable as well. Detergent wash solutions made from low conductivity wash buffers (<40 mS/cm) or neat water are particularly suited.

After loading the matrix is washed with at least one wash buffer. The wash buffer might be identical to the loading buffer or different from the loading buffer. The matrix might also be washed with 2, 3 or 4 different wash buffers. Optionally one of the wash buffers comprises a non-ionic detergent selected from the group of alkylglycosides and secondary alcohol alkoxylates or mixtures thereof. Typically, the concentration of the non-ionic detergent in the wash buffer, if present, is between 0.01% and 10% (w/v), preferably between 0.1% and 1.5% (w/v).

If a non-ionic detergent is added to a wash buffer, it is preferably added to the first wash buffer and in any case at least one further wash step is performed after the wash with the wash buffer comprising the detergent. Preferably the matrix is washed with more than one wash buffer.

In another preferred embodiment, one wash buffer, preferably the last wash buffer comprises ethanol in a concentration between 10 and 25% (v/v).

Preferably the pH and the ionic strength of the wash buffers is identical or similar to the pH and the ionic strength of the equilibration/loading buffer.

Elution of the target nucleic acids is then done by using an elution buffer. The elution buffer has a different pH and/or different ionic strength than the equilibration/loading buffer.

In one embodiment it has a higher pH and/or a higher ionic strength than the equilibration/loading buffer. In one embodiment the pH of the elution buffer is above pH 7, preferably between pH 8.5 and 9.5. In one embodiment the elution buffer comprises between 0.5 and 1.5 M NaCl. In any case, in the method of the present invention, at least one time a non-ionic detergent selected from the group of alkylglycosides and secondary alcohol alkoxylates or mixtures thereof is added. As described above, the detergent can be added to the feed and/or to a wash buffer.

Preferably the detergent is an alkylglycoside, most preferred a C8-C16 alkylglycoside, especially preferred the detergent is Triton® CG110.

By performing the method of the present invention, the target nucleic acid can be obtained with significantly lower endotoxin contamination compared to the contamination in the sample loaded onto the chromatography matrix.

The final endotoxin level in the target nucleic acid depends on the initial endotoxin level. With initial endotoxin levels around 1.3 10⁶ EU/mg target nucleic acid, with the method of the present invention final endotoxin levels of below 30 EU/mg target nucleic acid can be achieved. With initial endotoxin levels around 50.000 EU/mg target nucleic acid, with the method of the present invention final endotoxin levels of below 10 EU/mg target nucleic acid can be achieved.

It was found that the method of the present invention shows typically better results compared to results achieved by performing the same method with other detergents that are typically recommended for bead based applications like Triton® X100 and Tween® 80 or Tween®20.

In one embodiment, the method of the present invention is performed by only using one or more non-ionic detergents selected from the group of alkylglycosides and secondary alcohol alkoxylates or mixtures thereof. No other detergent is added to the feed or the wash buffers or at any other time during the chromatographic purification.

In one embodiment of the present invention the method further comprises an additional step for detecting residual endotoxin in the nucleic acids resulting from the chromatographic purification. It is typically of high relevance to check the quality of the nucleic acid product prior to further use. As endotoxins can cause unwanted side effects, controlling their removal or depletion is often crucial. A person skilled in the art is aware of methods for detecting endotoxins.

Limulus-based detection assays, LAL tests, are commonly regarded as state of the art analytical in-vitro detection method for endotoxins. Details about the LAL assay and other methods for detecting and measuring endotoxins are knows to a person skilled in the art. An example of an alternative method beside the LAL assay are recombinant factor based endotoxin detection kits like the recombinant factor C assays from Lonza. Further information can be found in E. C. Dullah, “Current trends in endotoxin detection and analysis of endotoxin-protein interactions”, February 2016, Critical Reviews in Biotechnology 37(2):1-11.

A severe interference of the LAL assay as well as other endotoxin assays like the recombinant factor based assays results from substances interacting with endotoxins forming “stealth” structures that shield the analyte from the LAL enzyme or the recombinant enzyme, an effect resulting in low endotoxin recovery (LER) and underestimation of the actual endotoxin concentration.

Detergents are well known to affect the detectability of endotoxins by forming micellar structures.

With the same amphiphilic nature and similar structure, both are considered perfect partners to interact. Consequently, care must be taken when analyzing endotoxins in final eluate samples since occurrence of LER-effects caused by residual detergent must be excluded.

It has been found that endotoxin assays like the LAL assay can be performed without the occurrence of LER effects with products obtained with the method of the present invention. Unexpectedly, the detergents used in the method of the present invention do not cause LER effects. Especially alkylglycosides and 2-ethyl hexanol ethylene oxide-propylene oxide copolymers do not show any interference even when present in high concentrations.

Consequently, in one embodiment the method of the present invention comprises an additional step for detecting residual endotoxin in the nucleic acids resulting from the chromatographic purification directly in the eluate of the chromatography matrix without any further treatment of the eluate.

The present invention is further illustrated by the following figures and examples, however, without being restricted thereto.

The entire disclosure of all applications, patents, and publications cited above and below as well as of the corresponding application U.S. 63/229,666 filed May 8, 2021, are hereby incorporated by reference.

### Examples

The following examples represent practical applications of the invention.

### List of Detergents

| | Active | Article number/ | |
|---|---|---|---|
| Chemical | [symbol] | [symbol] | Supplier |
| Tergitol@ 15-S-9 | ~100% | 15S9-100ML/ | Sigma |
| | | [symbol] | |
| Triton ® X100 | ~100% | 1.12298.1001/ | Merck |
| | | [symbol] | |
| Triton ® CG110 | ~60% | STS0005/ | Sigma |
| | | [symbol] | |
| EcoSurf ® EH 9 | ~100% | STS0006/ | Sigma |
| | | [symbol] | |
| Tween ® 20 | ~100% | P9416/ | Sigma |
| | | [symbol] | |
| Tween ® 80 | ~100% | 2.73536/ | NOF |
| | | [symbol] | |

[symbol]

indicates data missing or illegible when filed

### Protocols for Plasmid DNA Capture

Note 1: For each set of experiments testing individual detergents either as wash or feed supplement, a new membrane device was used to avoid artificial effects from cross contamination by carrying over of residual detergent between successive runs/experiments.

Note 2: Small volume monolith or membrane screening devices where the system holdups are disproportionately large, very high volumes were used for wash 1, wash 2, elution, cleaning in place (CIP) and equilibration are typical. At larger scale these values can be reduced and flow direction reversed for enhancing individual steps. This is a standard practice and common knowledge for any one proficient in art.

### Chromatography Materials

| Material (Device) | Article number | Supplier |
|---|---|---|
| Natrix ® Q Recon Mini | NXF-01 | Merck |
| with 0.2 mL membrane bed | | Millipore |
| volume | | |
| Mustang ® Q Acrodisc ® | MSTG25Q6 | Pall Life |
| with 0.18 mL membrane bed | | Sciences |
| volume | | |
| CIMmultus ® DEAE | 311.5114-2 | BIA |
| 1 mL packed column volume | | Separations |

Natrix® Q Protocol 1—Capture from Feed a (20 kb Plasmid) after Detergent Treatment

### Buffers

| Step | Buffer |
|---|---|
| equilibrate | 1M K-acetate + 100 mM NaCl, pH 5.0 |
| sample loading | 20 kb Plasmid DNA Lysate w/o detergent |
| | or |
| | 20 kb Plasmid DNA Lysate w/neutral detergent |
| | at specified concentration |
| wash 1 | 1M K-acetate + 100 mM NaCl, pH 5.0 |
| wash 2 | 20% EtOH + 150 mM NaCl |
| elution | 1.5M NaCl + 100 mM Tris, pH 8.0 |
| CIP | 2M NaCl + 1M NaOH |
| sample pump | 1M K-acetate + 100 mM NaCl, pH 5.0 |
| wash | |

### Chromatography Method

| Step | Volume | Flow rate | |
|---|---|---|---|
| equilibration | 5 MV (membrane volume) | 1 | m [symbol] /min |
| load | variable | 1 | m [symbol] /min |
| wash 1 | 20 MV | 1 | m [symbol] /min |
| wash 2 | 20 MV | 1 | m [symbol] /min |
| elution | 30 MV | 1 | mL/min |
| | plasmid eluate fraction: | | |
| | 0 −> 25 [symbol] | | |
| CIP | 20 MV −> 15 min | 1 | m [symbol] /min |
| | pause −> 20 MV [symbol] | | |
| equilibration | 40 MV | 1 | m [symbol] /min |

[symbol]

indicates data missing or illegible when filed

### Plasmid Feed

Original 20 kb Plasmid lysate used as feed showed an initial Endotoxin level of ˜1,300,000 EU/mg Plasmid. Plasmid lysate filtered with 0.22 μm PES media was supplemented with 100 mM NaCl required for selective binding of pDNA. Prior to Plasmid capture with Natrix® Q, a defined amount of detergent was added to the lysate. Following gentle stirring at room temperature for 30 min until detergent was completely dissolved and homogeneity of the mixture reached, the sample was subsequently subjected to purification experiments.

Natrix® Q Protocol 2—Capture from Feed B (8 kb Plasmid) after Detergent Treatment

### Chromatography Buffers

| Step | Buffer |
|---|---|
| equilibrate | 1M K-acetate + 160 mM NaCl, pH 5.0 |
| sample | 8 kb Plasmid DNA Lysate w/o detergent |
| loading | or |
| | 8 kb Plasmid DNA Lysate w/neutral detergent at |
| | specified concentration |
| wash 1 | 1M K-acetate + 160 mM NaCl, pH 5.0 |
| wash 2 | 20% EtOH + 150 mM NaCl |
| elution | 1M NaCl + 100 mM Tris, pH 9.0 |
| CIP | 2M NaCl + 1M NaOH |
| sample | 1M K-acetate + 160 mM NaCl, pH 5.0 |
| pump wash | |

### Chromatography Method

| Step | Volume | Flow rate |
|---|---|---|
| equilibration | 5 MV | 2 mL/min |
| load | variable | 2 mL/min |
| wash 1 | 20 MV | 2 mL/min |
| wash 2 | 20 MV | 1 mL/min |
| elution | 30 MV | 1 mL/min |
| | plasmid eluate fraction: | |
| | 0 −> 25 MV | |
| CIP | 20 MV −> 15 min | 2 mL/min |
| | pause −> 20 MV | |
| equilibration | 40 MV | 2 mL/min |

### Plasmid Feed

Original 8 kb Plasmid lysate used as feed showed an initial Endotoxin level of ˜50,000 EU/mg Plasmid. Plasmid lysate filtered with 0.22 μm PES media was supplemented with 175 mM NaCl required for selective binding of pDNA. Prior to Plasmid capture with Natrix Q, a defined amount of detergent was added. Following gentle stirring at room temperature for 30 min until detergent was completely dissolved and homogeneity of the mixture reached, the sample was subsequently subjected to purification experiments.

Natrix® Q Protocol 3—Capture from Feed C (8 kb Plasmid) Using Neutral Detergent Wash

### Chromatography Buffers

| Step | Buffer |
|---|---|
| equilibrate | 1M K-acetate + 160 mM NaCl, pH 5.0 |
| sample loading | 8 kb Plasmid DNA Lysate |
| | w/o detergent |
| wash 1 | 1M K-acetate + 160 mM NaCl, pH 5.0 |
| | or |
| | 1M K-acetate + 160 mM NaCl, pH 5.0 |
| | w/neutral detergent at specified concentration |
| wash 2 | 1M K-acetate + 160 mM NaCl, pH 5.0 |
| wash 3 | 20% EtOH + 150 mM NaCl |
| elution | 1M NaCl + 100 mM Tris, pH 9.0 |
| CIP | 2M NaCl + 1M NaOH |
| sample pump | 1M K-acetate + 160 mM NaCl, pH 5.0 |
| wash | |

### Chromatography Method

| Step | Volume | Flow rate | |
|---|---|---|---|
| equilibration | 5 MV | 2 | m [symbol] /min |
| load | variable | 2 | m [symbol] /min |
| wash 1 | 20 MV | 2 | m [symbol] /min |
| wash 2 | 20 MV | 2 | m [symbol] /min |
| wash 3 | 20 MV | 1 | m [symbol] /min |
| elution | 30 MV | 1 | mL/min |
| | plasmid eluate fraction: | | |
| | 0 −> [symbol] | | |
| CIP | 20 MV −> 15 min | 2 | m [symbol] /min |
| | pause −> 20 [symbol] | | |
| equilibration | 40 MV | 2 | m [symbol] /min |

[symbol]

indicates data missing or illegible when filed

### Plasmid Feed

Original 8 kb Plasmid lysate used as feed showed an initial Endotoxin level of ˜275,000 EU/mg Plasmid. The lysate filtered with 0.22 μm PES media and supplemented with 175 mM NaCl required for selective binding of pDNA.

Mustang® Q Protocol 1—Capture from Feed C (8 kb Plasmid) Using Neutral Detergent Wash

### Chromatography Buffers

| Step | Buffer |
|---|---|
| equilibrate | 1M K-acetate + 375 mM NaCl, pH 5.0 |
| sample loading | 8 kb Plasmid DNA Lysate |
| | w/o detergent |
| wash 1 | 1M K-acetate + 375 mM NaCl, pH 5.0 |
| | or |
| | 1M K-acetate + 375 mM NaCl, pH 5.0 |
| | w/detergent at specified concentration |
| wash 2 | 1M K-acetate + 375 mM NaCl, pH 5.0 |
| wash 3 | 20% EtOH + 150 mM NaCl |
| elution | 1M NaCl + 100 mM Tris, pH 9.0 |
| CIP | 2M NaCl + 1M NaOH |
| sample pump | 1M K-acetate + 375 mM NaCl, pH 5.0 |
| wash | |

### Chromatography Method

| Step | Volume | Flow rate |
|---|---|---|
| equilibration | 5 MV | 1.8 mL/min |
| load | variable | 1.8 mL/min |
| wash 1 | 22 MV | 1.8 mL/min |
| wash 2 | 22 MV | 1.8 mL/min |
| wash 3 | 22 MV | 1.8 mL/min |
| elution | 33 MV | 0.9 mL/min |
| | plasmid eluate fraction: | |
| | 0 −> 28 MV | |
| CIP | 22 MV −> 15 min | 1.8 mL/min |
| | pause −> 22 MV | |
| equilibration | 40 MV | 1.8 mL/min |

### Plasmid Feed

Purification trials were conducted with original lysate filtered with 0.22 μm PES media and supplemented with 375 mM NaCl required for selective binding of pDNA.

CIMmultus® DEAE Protocol 1—Capture from Feed C (8 kb Plasmid) Using Neutral Detergent Wash

### Chromatography Buffers

| Step | Buffer |
|---|---|
| equilibrate | 1M K-acetate + 60 mM NaCl, pH 5.0 |
| sample loading | 8 kb Plasmid DNA Lysate |
| | w/o detergent |
| wash 1 | 1M K-acetate + 60 mM NaCl, pH 5.0 |
| | or |
| | 1M K-acetate + 60 mM NaCl, pH 5.0 |
| | w/detergent at specified concentration |
| wash 2 | 1M K-acetate + 60 mM NaCl, pH 5.0 |
| wash 3 | 20% EtOH + 150 mM NaCl |
| elution | 1M NaCl + 100 mM Tris, pH 9.0 |
| CIP | 2M NaCl + 1M NaOH |
| sample pump | 1M K-acetate + 60 mM NaCl, pH 5.0 |
| wash | |

### Chromatography Method

| Step | Volume | Flow rate |
|---|---|---|
| equilibration | 3 CV (column volume) | 5 mL/min |
| load | variable | 5 mL/min |
| wash 1 | 5 CV | 5 mL/min |
| wash 2 | 5 CV | 5 mL/min |
| wash 3 | 5 CV | 5 mL/min |
| elution | 10 CV | 5 mL/min |
| | plasmid eluate fraction: | |
| | 0 −> 5 MV | |
| CIP | 3 MV −> 10 min | 5 mL/min |
| | pause −> 2 MV | |
| equilibration | 5 MV | 5 mL/min |

### Plasmid Feed

Purification trials were conducted with original lysate filtered with 0.22 μm PES media and supplemented with 60 mM NaCl required for selective binding of pDNA.

### Endotoxin Assay

Endotoxin analytic was conducted using the cartridge-based Limulus Amebocyte Lysate (LAL) Endosafe-PTS system from Charles River following manufacturer's instructions.

### Plasmid DNA Analytics

Purity and Quantity and Plasmid DNA in original lysate and samples collected from Natrix® Q capture trials were determined my means of analytical UV/HPLC method.

### Detergents Analytics

Residual amount of detergent in Plasmid eluate fractions collected from Natrix® Q capture trials was measured by means of an analytical HPLC method as described below. The method allows for direct analysis of Plasmid eluate samples without prior sample preparation for removing potentially interfering matrix components by means of e.g. solid phase extraction.

The amount of detergent in unknown eluate samples was calculated based on the analyte peak area using calibration curves obtained from standards of individual detergents in eluate buffer matrix.

The compatibility of analytical method for real plasmid samples and validity of analytical results was demonstrated by means of spike-recovery tests. For that purpose, the recovery of a defined amount of individual detergent spiked into Plasmid eluate samples (from capture trials without use of any detergents) was verified.

| Column type: | Oasis HLB, 25 μm 2.1 × 20 mm, | | |
|---|---|---|---|
| | from Waters, Cat. No. 186002036 | | |
| Column dimensions: | 2.1 × 20 mm (ID × L) | | |
| Guard column: | N/A | | |
| Eluent A: | water + 0.1% v/v formic acid | | |
| Eluent B: | Acetonitrile + 0.1% v/v formic acid | | |
| Injection volume: | 5-100 μL | | |
| Flow rate: | 1 ml/min | | |
| Pressure: | max. 250 bar | | |
| Detection: | Evaporate Light Scattering Detector (ELSD) | | |
| Gradient: | time (min) | % A | % B |
| | 0.0 | 95 | 5 |
| | 5.5 | 95 | 5 |
| | 6.0 | 0 | 100 |
| | 8.0 | 0 | 100 |
| | 8.1 | 95 | 5 |
| | 11.0 | 95 | 5 |

Test A—Lipopolysaccharide (LPS) Spike Detection in Eluate Buffers with Detergent

This test was conducted using lyophilized E. coli (O111:B4) Endotoxin Standard from Thermo Scientific (Cat #1897398). LPS standard was reconstituted in water yielding a nominal concentration of 50 EU/mL.

### Part 1) Spike-Recovery in 1.5 M NaCl+100 mM Tris pH 8.0 Buffer Eluate Buffer

Detectability of LPS in Plasmid eluate buffer matrix (1.5M NaCl+100 mM Tris/HCl, pH 8.0) with residual amount of detergent was tested according to following protocol:
Lyophilized LPS standard was dissolved in water yielding an LPS stock solution with nominal 50 EU/mL Endotoxin.
20 μL LPS stock solution was mixed with 100 μL of eluate buffer supplemented with varying amount of different detergent.
Mixture was incubated at 30° C. for 1 hour, afterwards shortly centrifuged and mixed prior to final dilution by adding 880 μL of water.
Samples were directly analyzed for Endotoxin using 5-0.05 EU/mL cartridges and the results of recovered spike evaluated.

### Part 2) Spike-Recovery in 1 M NaCl+100 mM Tris pH 9.0 Buffer Eluate Buffer

Detectability of LPS in Plasmid eluate buffer matrix (1 M NaCl+100 mM Tris/HCl, pH 9.0) with residual amount of detergent was tested according to following protocol:
Lyophilized LPS standard was dissolved in water yielding an LPS stock solution with nominal 50 EU/mL Endotoxin.
20 μL LPS stock solution was mixed with 100 μL of eluate buffer supplemented with varying amount of different detergent.
Mixture was incubated at 30° C. for 1 hour, afterwards shortly centrifuged and mixed prior to final dilution by adding 880 μL of 25 mM Tris/HCl buffer, pH 7.0.
Samples were directly analyzed for Endotoxin using 5-0.05 EU/mL cartridges and the results of recovered spike evaluated.

### Test B—Residual Endotoxin Detection in Plasmid Eluate in Presence of Tergitol®

In this experiment the recovery of Endotoxins in real Plasmid eluate samples in presence of defined amount of Tergitol® 15-S-9 was investigated. Samples of Plasmid eluate material obtained from a Natrix® Q capture run conducted without use of any detergent were subsequently spiked with defined amount of detergent and finally analyzed for Endotoxin.
Starting material was an 8 kb Plasmid eluate pool w/o detergent showing an Endotoxin level of ˜200 EU/mL. Eluate buffer matrix was equivalent to 1 M NaCl+100 mM Tris, pH 9.0.
Detergent stock solutions were prepared in eluate buffer 1 M NaCl+100 mM Tris, pH 9.0.
Pipetting scheme for preparation of spike samples was as follows:

| Tergitol ® | | | |
|---|---|---|---|
| Spike in ppm^{†} | Sample Mix | | |
| 0 | 500 μL | + | 50 μL |
| | pDNA eluate | | eluate buffer |
| 9 | 500 μL | + | 50 μL |
| | pDNA eluate | | 100 ppm Tergitol@ stock |
| 45 | 500 μL | + | 50 μL |
| | pDNA eluate | | 500 ppm Tergitol@ stock |

^{†}Tergitol ® concentration (active substance) in final sample mix

Mixtures were incubated at 30° C. for 5 min, afterwards shortly centrifuged and mixed.
Prior to analytics samples were diluted with 25 mM Tris/HCL buffer, pH 7.0 by factor 204 and subjected to Endotoxin measurement using 5-0.05 EU/mL test cartridges.
   Test C—Detection of Endotoxin in Plasmid Eluate Spiked with 200 Ppm of Neutral Detergent

In this experiment the recovery of Endotoxins in real Plasmid eluate samples in presence of defined amount of neutral detergent was investigated. Samples of Plasmid eluate material obtained from a Natrix® Q capture run conducted without use of any detergent were subsequently spiked with a defined amount of detergent and finally analyzed for Endotoxin.
Starting material was a 20 kb Plasmid eluate pool w/o detergent showing an Endotoxin level of ˜500 EU/mL. Eluate buffer matrix was equivalent to 1.5 M NaCl+100 mM Tris, pH 8.0.
Detergent stock solutions at 10.000 ppm were prepared in water.
Pipetting scheme for preparation of spike samples was as follows:

| Detergent Spike^{†} | Sample Preparation | | |
|---|---|---|---|
| w/o | 490 μL | + | 10 μL |
| | pDNA eluate | | water |
| Triton ® X100 | 490 μL | + | 10 μL |
| 200 ppm | pDNA eluate | | 10.000 ppm Triton ® X100 |
| | | | stock |
| Triton ® CG110 | 490 μL | + | 10 μL |
| 120 ppm | pDNA eluate | | 6.000 ppm active Triton ® |
| | | | CG110 stock (active substance) |
| EcoSurf ® EH 9 | 490 μL | + | 10 μL |
| 200 ppm | pDNA eluate | | 10.000 ppm EcoSurf ® |
| | | | EH-9 stock |
| Tween ® 20 | 490 μL | + | 10 μL |
| 200 ppm | pDNA eluate | | 10.000 ppm Tween ® 20 |
| | | | stock |
| Tween ® 80 | 490 μL | + | 10 μL |
| 200 ppm | pDNA eluate | | 10.000 ppm Tween ® 80 |
| | | | stock |
| Tergitol ® 15-S-9 | 490 μL | + | 10 μL |
| 200 ppm | pDNA eluate | | 10.000 ppm Tergitol ® |
| | | | 15-S-9 stock |

^{†}detergent concentration (active substance) in final sample mix

490 μL of Plasmid eluate was mixed with 10 μL of corresponding detergent stock solution.
Mixtures were incubated over night at 8° C. for 16 h, then incubated at 30° C. for 5 min, afterwards shortly centrifuged and mixed.
Prior to analytics samples were diluted with water by factor 1,000 and finally subjected to Endotoxin measurement using 5-0.05 EU/mL test cartridges.

### Results

1) Plasmid Capture with Natrix® Q from Feed A (20 kb pDNA) Treated with Detergent

Tables R1 (Parts A and B) and R2 compare results obtained from Plasmid DNA capture trials with Natrix® 0 testing different detergents for lysate pre-treatment according to the Natrix Q protocol 1. The membrane loading was 0.5 mg Plasmid/mL membrane volume. Original 20 kb Plasmid lysate used as feed showed an initial Endotoxin level of ˜1,300,000 EU/mg Plasmid.

TABLE R1
Part A: Analytical data for 20 kb Plasmid purification
testing various detergents as feed supplement

| | Active | | Eluate Endotoxin | | Residual | |
|---|---|---|---|---|---|---|
| | Substance | | Level in | | Detergent in | |
| Feed | Concentration | | EU/mg pDNA | | pDNA Pool in mg/L | |
| Supplement | in the Feed | | Run1 | Run2 | Run 1 | Run 2 |
| w/o | —/— | | 10,550 | 28,846 | NA | NA |
| w/o | —/— | | 25,169 | 34,407 | NA | NA |
| w/o | —/— | | 31,443 | 41,988 | NA | NA |
| Triton ® X100 | 1% | w/v | 568 | 444 | 76 | 197 |
| Tween ® 80 | 1% | w/v | 19,791 | 23,751 | 41 | 45 |
| Tween ® 20 | 1% | w/v | 22,180 | 25,195 | 60 | 64 |
| EcoSurf ® | 1% | w/v | 169 | 359 | 394 | 34 |
| EH9 | | | | | | |
| Triton ® | 0.6% | w/v | <28 | 124 | 98 | 104 |
| CG110 | | | | | | |

TABLE R1
Part B: Analytical data for 20 kb Plasmid purification
testing various detergents as feed supplement

| | Active Substance | | Plasmid Eluate | | Plasmid Eluate Purity | |
|---|---|---|---|---|---|---|
| Feed | Concentration | | Yield (%) | | in % (total A260 area) | |
| Supplement | in the Feed | | Run 1 | Run2 | Run 1 | Run2 |
| w/o | —/— | | 76 | 71 | ~66 | ~60 |
| w/o | —/— | | 73 | 73 | ~50 | ~50 |
| w/o | —/— | | 84 | 77 | ~45 | ~44 |
| Triton ® | 1% | w/v | 85 | 71 | ~31 | ~31 |
| X100 | | | | | | |
| Tween ® 80 | 1% | w/v | 83 | 76 | ~62 | ~67 |
| Tween ® 20 | 1% | w/v | 74 | 83 | ~44 | ~62 |
| EcoSurf ® | 1% | w/v | 90 | 78 | ~70 | ~54 |
| EH9 | | | | | | |
| Triton ® | 0.6% | w/v | 87 | 77 | ~45 | ~28 |
| CG110 | | | | | | |

The efficacy of endotoxin removal observed with different detergents tested as supplement for feed pre-treatment is given in Table R2.

TABLE R2
Factor of Endotoxin reduction in Plasmid eluate pools
relative to the baseline experiment conducted without
use of detergent. Values stated are mean values calculated
from duplicate runs and repetition trials.

| Feed | Active Substance | | Endotoxin |
|---|---|---|---|
| Supplement | Concentration in the Feed | | Reduction Factor |
| Triton ® X100 | 1% | w/v | 57 |
| Tween ® 80 | 1% | w/v | 1 |
| Tween ® 20 | 1% | w/v | 1 |
| EcoSurf ® EH9 | 1% | w/v | 109 |
| Triton ® CG110 | 0.6% | w/v | 383 |

2) Plasmid Capture with Natrix® Q from Feed B (8 kb pDNA) Treated with Detergent

Table R3 (Part A and B) and Table R4 compare results obtained from Plasmid DNA capture trials with Natrix® Q testing different detergents for lysate pre-treatment according to the Natrix Q protocol 2.

The membrane loading was 1.6 mg Plasmid/mL membrane volume. Original 8 kb Plasmid lysate used as feed showed an initial Endotoxin level of ˜ 50,000 EU/mg Plasmid.

TABLE R3
Part A. Analytical data for 8 kb Plasmid purification
testing various detergents as wash buffer supplement

| | Active | | Eluate Endotoxin | | Residual | |
|---|---|---|---|---|---|---|
| | Substance | | Level in | | Detergent in | |
| Feed | Concentration | | EU/mg pDNA | | pDNA Pool in mg/L | |
| Supplement | in the Feed | | Run1 | Run2 | Run 1 | Run 2 |
| w/o | —/— | | 388 | 777 | N/A | N/A |
| Triton ® X100 | 1% | w/v | 25 | 26 | 126 | 116 |
| Tween ® 80 | 1% | w/v | 302 | 331 | 53 | 57 |
| Tween ® 20 | 1% | w/v | 599 | 580 | 80 | 89 |
| EcoSurf ® | 1% | w/v | 49 | 50 | 102 | 91 |
| EH9 | | | | | | |
| Triton ® | 0.6% | w/v | <9 | <16 | 35 | 52 |
| CG110 | | | | | | |

TABLE R3
Part B. Analytical data for 8 kb Plasmid purification
testing various detergents as wash buffer supplement

| | Active Substance | | Plasmid Eluate | | Plasmid Eluate Purity | |
|---|---|---|---|---|---|---|
| Feed | Concentration | | Yield (%) | | in % (total A260 area) | |
| Supplement | in the Feed | | Run 1 | Run2 | Run 1 | Run2 |
| w/o | —/— | | 90 | 90 | ~86 | ~86 |
| Triton ® | 1% | w/v | 93 | 95 | ~74 | ~74 |
| X100 | | | | | | |
| Tween ® 80 | 1% | w/v | 89 | 96 | ~81 | ~78 |
| Tween ® 20 | 1% | w/v | 91 | 94 | ~80 | ~77 |
| EcoSurf ® | 1% | w/v | 92 | 95 | ~78 | ~84 |
| EH9 | | | | | | |
| Triton ® | 0.6% | w/v | 85 | 86 | ~82 | ~82 |
| CG110 | | | | | | |

The efficacy of endotoxin removal observed with different detergents tested as wash buffer supplement is given in Table R4.

TABLE R4
Factor of Endotoxin reduction in Plasmid eluate pools
relative to the baseline experiment conducted without
use of detergent. Values stated are mean values calculated
from duplicate runs and repetition trials.

| | | |
|---|---|---|
| | Active Substance | Endotoxin |
| | Concentration | Reduction |
| Feed Supplement | in the Feed | Factor |

| | | | |
|---|---|---|---|
| Triton ® X100 | 1% | w/v | 23 |
| Tween ® 80 | 1% | w/v | 2 |
| Tween ® 20 | 1% | w/v | 1 |
| EcoSurf ® EH9 | 1% | w/v | 12 |
| Triton ® CG110 | 0.6% | w/v | >45 |

3) Plasmid Capture with Natrix Q from Feed C (8 kb pDNA) Testing Detergent Wash Buffers

Tables R5 (Parts A and B) and R6 compare results obtained from Plasmid DNA capture trials with Natrix Q testing different detergents as wash buffer supplement according to the Natrix Q protocol 3. The membrane loading was 1.6 mg Plasmid/mL membrane volume. Original 8 kb Plasmid lysate used as feed showed an initial Endotoxin level of ˜275,000 EU/mg Plasmid.

TABLE R5
Part A. Analytical data for 8 kb Plasmid eluate
pools obtained with different wash buffers

| | Active | | | | Residual | |
|---|---|---|---|---|---|---|
| | Substance | | Eluate Endotoxin | | Detergent | |
| | Concentration | | Level in | | in pDNA Pool | |
| Feed | in Wash | | EU/mg pDNA | | in mg/L | |
| Supplement | Buffer | | Run1 | Run2 | Run 1 | Run 2 |
| w/o | —/— | | 2,917 | 6,955 | N/A | N/A |
| Triton ® X100 | 1% | w/v | 43 | 71 | 19 | 18 |
| Tween ® 80 | 1% | w/v | 9,159 | 7,242 | 15 | 15 |
| Tween ® 20 | 1% | w/v | 7,328 | 9,800 | 22 | 23 |
| EcoSurf ® EH9 | 1% | w/v | 55 | 100 | 13 | 13 |
| Triton ® CG110 | 0.6% | w/v | 10 | 10 | 13 | 34 |
| Triton ® CG110 | 1% | w/v | 4.2 | 1.5 | 26 | 27 |
| Triton ® CG110 | 2.4% | w/v | <1.7 | <1.6 | 97 | 93 |
| Tergitol ® | 1% | w/v | 76 | 86 | 18 | 12 |
| 15-S-9 | | | | | | |

TABLE R5
Part B. Analytical data for 8 kb Plasmid eluate
pools obtained with different wash buffers

| | Active | | | | | |
|---|---|---|---|---|---|---|
| | Substance | | Plasmid Eluate | | Plasmid Purity | |
| | Concentration | | Yield | | in % (total | |
| Feed | in Wash | | (%) | | A260 area) | |
| Supplement | Buffer | | Run 1 | Run2 | Run 1 | Run2 |
| w/o | —/— | | 88 | 95 | ~94 | ~91 |
| Triton X100 | 1% | w/v | 93 | 94 | ~90 | ~93 |
| Tween ® 80 | 1% | w/v | 88 | 90 | ~92 | ~92 |
| Tween ® 20 | 1% | w/v | 89 | 93 | ~87 | ~90 |
| EcoSurf ® EH9 | 1% | w/v | 94 | 94 | ~92 | ~94 |
| Triton ® CG110 | 0.6% | w/v | 88 | 97 | ~88 | ~90 |
| Triton ® CG110 | 1% | w/v | 90 | 97 | ~96 | ~96 |
| Triton ® CG110 | 2.4% | w/v | 87 | 90 | ~95 | ~95 |
| Tergitol@ 15-S-9 | 1% | w/v | 93 | 94 | ~90 | ~94 |

The efficacy of endotoxin removal observed with different detergents tested as wash buffer supplement is given in Table R6.

TABLE R6
Factor of Endotoxin reduction in Plasmid eluate pools relative
to the baseline experiment conducted without use of detergent.
Values stated are mean values calculated from duplicate
runs and, respectively, repetition trials.

| | | |
|---|---|---|
| | Active Substance | Endotoxin |
| | Concentration | Reduction |
| Feed Supplement | in Wash Buffer | Factor |

| | | | |
|---|---|---|---|
| Triton ® X100 | 1% | w/v | 87 |
| Tween ® 80 | 1% | w/v | 1 |
| Tween ® 20 | 1% | w/v | 1 |
| EcoSurf ® EH9 | 1% | w/v | 64 |
| Triton ® CG110 | 0.6% | w/v | 494 |
| Triton ® CG110 | 1% | w/v | 1732 |
| Triton ® CG110 | 2.4% | w/v | >2992 |
| Tergitol@ 15-S-9 | 1% | w/v | 61 |

Residual host cell protein concentration in Plasmid eluate pools obtained from Natrix Q capture runs conducted with different detergent wash buffers are listed in Table 7. Results suggest for lowest HCP impurity levels in Plasmid eluate pools from purification protocols based on use of Triton CG110.

TABLE R7
Removal of E. coli host cell protein (HCP) from Plasmid
DNA during Natrix Q capture step. Table below compares
residual HCP concentrations measured in Plasmid eluate
pools from Natrix Q capture following different detergent
wash protocols. For each wash protocol, plasmid eluate
pools collected from two consecutive runs were analyzed
(referred as run1 and run 2). HCP concentration in original
Plasmid lysate amounted to 3,235 μg HCP/mg pDNA.

| | | |
|---|---|---|
| | Active | Residual HCP conc. |
| Wash Buffer | Substance in | in ng/mg pDNA |

| Supplement | Wash Buffer | | Run 1 | Run 2 |
|---|---|---|---|---|
| w/o | —/— | | 439 | 1000 |
| Triton ® X100 | 1% | w/v | 4213 | 2129 |
| Tween ® 80 | 1% | w/v | 6471 | 2967 |
| Tween ® 20 | 1% | w/v | 8483 | 4377 |
| EcoSurf ® EH9 | 1% | w/v | 1629 | 1065 |
| Triton ® CG110 | 0.6% | w/v | 193 | 339 |
| Triton ® CG110 | 1% | w/v | 1097 | 952 |
| Triton ® CG110 | 2.4% | w/v | 1766 | 1687 |
| Tergitol ® 15-S-9 | 1% | w/v | 2222 | 561 |

4) Plasmid Capture with Mustang® Q from Feed C (8 kb pDNA) Testing Detergent Wash Buffers

Tables R8 (Parts A and B) and R7 compare results obtained from Plasmid DNA capture trials with Mustang® Q testing different detergents as wash buffer supplement according to the Mustang® Q protocol 1. The membrane loading was 1.6 mg Plasmid/mL membrane volume. Original 8 kb Plasmid lysate used as feed showed an initial Endotoxin level of ˜275,000 EU/mg Plasmid.

TABLE R8
Part A. Analytical data for 8 kb Plasmid eluate
pools obtained with different wash buffers

| | Active | | | | Residual | |
|---|---|---|---|---|---|---|
| | Substance | | Eluate Endotoxin | | Detergent in | |
| | Concentration | | Level in | | pDNA Pool | |
| Feed | in Wash | | EU/mg pDNA | | in mg/L | |
| Supplement | Buffer | | Run1 | Run2 | Run 1 | Run 2 |
| w/o | —/— | | 3,983 | 7,081 | N/A | N/A |
| Triton ® X100 | 1% | w/v | 116 | 130 | 38 | 34 |
| Triton ® CG110 | 0.6% | w/v | 17 | 28 | 6 | 6 |

TABLE R8
Part B. Analytical data for 8 kb Plasmid eluate
pools obtained with different wash buffers

| | Active | | | | | |
|---|---|---|---|---|---|---|
| | Substance | | Plasmid Eluate | | Plasmid Purity | |
| | Concentration | | Yield | | in % (total | |
| Feed | in Wash | | (%) | | A260 area) | |
| Supplement | Buffer | | Run 1 | Run2 | Run 1 | Run2 |
| w/o | —/— | | 32 | 33 | >95 | >95 |
| Triton ® X100 | 1% | w/v | 37 | 28 | >95 | >95 |
| Triton ® CG110 | 0.6% | w/v | 41 | 34 | >95 | >95 |

The efficacy of endotoxin removal observed with different detergents tested as wash buffer supplement is given in Table R9.

TABLE R9
Factor of Endotoxin reduction in Plasmid eluate pools relative
to the baseline experiment conducted without use of detergent.
Values stated are mean values calculated from duplicate
runs and, respectively, repetition trials.

| | | |
|---|---|---|
| | Active Substance | Endotoxin |
| | Concentration | Reduction |
| Feed Supplement | in Wash Buffer | Factor |

| | | | |
|---|---|---|---|
| Triton ® X100 | 1% | w/v | 45 |
| Triton ® CG110 | 0.6% | w/v | 246 |

Residual host cell protein concentration in Plasmid eluate pools obtained from Mustang® Q capture runs conducted with different detergent wash buffers are listed in Table 10. Results confirm improved HCP clearance using a Triton® CG110 wash protocol.

TABLE R10
Removal of E. coli host cell protein (HCP) from Plasmid
DNA during Mustang ® Q capture step. Table below
compares residual HCP concentrations measured in Plasmid eluate
pools from Mustang ® Q capture following different
detergent wash protocols. For each wash protocol, plasmid
eluate pools collected from two consecutive runs were analyzed
(referred as run1 and run 2). HCP concentration in original
Plasmid lysate amounted to 3,235 μg HCP/mg pDNA.

| | | |
|---|---|---|
| | Active | Residual HCP |
| | Substance | conc. |
| Wash Buffer | in Wash | in ng/mg pDNA |

| Supplement | Buffer | | Run 1 | Run 2 |
|---|---|---|---|---|
| w/o | —/— | | 2380 | 6457 |
| Triton ® X100 | 1% | w/v | 2814 | 2785 |
| Triton ® CG110 | 0.6% | w/v | 1303 | 2143 |

5) Plasmid Capture with CIMmultus® DEAE from Feed C (8 kb pDNA) Testing Detergent Wash Buffers

Tables R11 (Parts A and B) and R12 compare results obtained from Plasmid DNA capture trials with CIMmultus® DEAE testing different detergents as wash buffer supplement according to the CIMmultus® DEAE protocol 1. The column loading was ˜1 mg Plasmid/mL column volume. Original 8 kb Plasmid lysate used as feed showed an initial Endotoxin level of ˜275,000 EU/mg Plasmid.

TABLE R11
Part A. Analytical data for 8 kb Plasmid eluate
pools obtained with different wash buffers

| | Active | | | Residual | |
|---|---|---|---|---|---|
| | Substance | Eluate Endotoxin | | Detergent | |
| | Concentration | Level in | | in pDNA Pool | |
| Feed | in Wash | EU/mg pDNA | | in mg/L | |
| Supplement | Buffer | Run1 | Run2 | Run 1 | Run 2 |
| w/o | —/— | 970 | 1,063 | N/A | N/A |
| Triton ® X100 | 1% w/v | 9.7 | 13.0 | 12 | 12 |
| Triton ® CG110 | 1% w/v | 0.5 | 0.7 | 14 | 15 |

TABLE R10
Part B. Analytical data for 8 kb Plasmid eluate
pools obtained with different wash buffers

| | Active | | | | |
|---|---|---|---|---|---|
| | Substance | Plasmid Eluate | | Plasmid Purity | |
| | Concentration | Yield | | in % (total | |
| Feed | in Wash | (%) | | A260 area) | |
| Supplement | Buffer | Run 1 | Run2 | Run 1 | Run2 |
| w/o | —/— | 87 | 90 | ~92 | ~92 |
| Triton ® X100 | 1% w/v | 91 | 93 | ~96 | ~93 |
| Triton ® CG110 | 1% w/v | 91 | 92 | ~95 | ~94 |

The efficacy of endotoxin removal observed with different detergents tested as wash buffer supplement is given in Table R12.

TABLE R12
Factor of Endotoxin reduction in Plasmid eluate pools relative
to the baseline experiment conducted without use of detergent.
Values stated are mean values calculated from duplicate
runs and, respectively, repetition trials.

| | Active Substance | Endotoxin |
|---|---|---|
| | Concentration | Reduction |
| Feed Supplement | in Wash Buffer | Factor |
| Triton ® X100 | 1% w/v | 90 |
| Triton ® CG110 | 1% w/v | 1694 |

Residual host cell protein concentration in Plasmid eluate pools obtained from CIMmultus® DEAE capture runs conducted with different detergent wash buffers are listed in Table 13. Results confirm improved HCP clearance using a Triton® CG110 wash protocol.

TABLE R13
Removal of E. coli host cell protein (HCP) from Plasmid
DNA during CIMmultus ® DEAE capture step. Table
below compares residual HCP concentrations measured in Plasmid
eluate pools from CIMmultus ® DEAE capture following
different detergent wash protocols. For each wash protocol,
plasmid eluate pools collected from two consecutive runs
were analyzed (referred as run1 and run 2). HCP concentration
in original Plasmid lysate amounted to 3,235 μg HCP/mg pDNA.

| | | |
|---|---|---|
| | Active | Residual HCP |
| | Substance | conc. |
| Wash Buffer | in Wash | in ng/mg pDNA |

| Supplement | Buffer | Run 1 | Run 2 |
|---|---|---|---|
| w/o | —/— | 652 | 746 |
| Triton ® X100 | 1% w/v | 967 | 900 |
| Triton ® CG110 | 1% w/v | 599 | 562 |

### Endotoxin Assay Interference (Endotoxin Masking Effect)

Test A—Lipopolysaccharide (LPS) Spike Detection in Eluate Buffers with Detergent

Table R14 summarizes data on the recovery observed for LPS in eluate buffers with different detergents. Data indicate that occurrence of interference of the LAL-assay for the detection of LPS depends on the type and concentration of residual detergent.

TABLE R14
Spike-recovery of LPS different eluate buffers with varying levels of various detergents
according to protocols for detection of LPS as detailed above 20 μL LPS-stock solution
was spiked into 100 μL of eluate buffer A) 1.5M NaCl + 100 mM Tris, pH 8.0 or B)
1.0M NaCl + 100 mM Tris, pH 9.0 supplemented with different detergents at varying concentration.

| | LPS Spike Recovery* in % | | | | | | | | |
|---|---|---|---|---|---|---|---|---|---|
| Active | Triton ® | | Triton ® | EcoSurf ® | | Tween ® | Tween ® | Tergitol ® | |
| Substance | CG110 | | X100 | EH-9 | | 20 | 80 | 15-S-9 | |
| Conc. | Buffer | Buffer | Buffer | Buffer | Buffer | Buffer | Buffer | Buffer | Buffer |
| in ppm† | A | B | A | B | A | A | A | A | B |
| 417 | N/A | N/A | <8 | 5 | 88 | 90 | 116 | <7 | <3 |
| 250 | 109 | 95 | N/A | N/A | N/A | N/A | N/A | N/A | N/A |
| 83 | N/A | N/A | <8 | 25 | 91 | 167 | 136 | 23 | 7 |
| 50 | 149 | 100 | N/A | N/A | N/A | N/A | N/A | N/A | N/A |
| 8 | N/A | N/A | 117 | 116 | 70 | 133 | 154 | 94 | 99 |
| 5 | 89 | 107 | N/A | N/A | N/A | N/A | N/A | N/A | N/A |
| 0 | 100 | 100 | 100 | 100 | 100 | 100 | 100 | 100 | 100 |

†detergent † co† in final sample/spike mix

### Test B—Residual Endotoxin Detection in Plasmid Eluate in Presence of Tergitol®

Endotoxin recovery results from test B using real plasmid samples containing defined amounts of Tergitol® are given inError! Reference source not found. Table R15. Data show that in presence of Tergitol® at low concentrations up to 45 ppm Endotoxin detection is not interfered.

TABLE R15
Recovery of Endotoxin in real Plasmid eluates with defined
residual amount of Tergitol ® 15-S-9

| Tergitol ® Spike in | Endotoxin recovered in |
|---|---|
| ppm ^{‡} | EU/mL |
| 0 | 244 |
| 9 | 207 |
| 45 | 219 |

^{‡} active substance concentration in final sample mix

Test C—Detection of Endotoxin in Plasmid Eluate Spiked with 200 Ppm of Neutral Detergent

Endotoxin recovery results from test C using real plasmid samples containing neutral detergent are given in Table R16. Severely impaired Endotoxin recovery was found for Triton® X100.

TABLE R16
Recovery of Endotoxin in real Plasmid eluates with
defined residual amount of neutral detergents.

| Detergent Spike ^{‡} | Spike Recovery in % |
|---|---|
| w/o | 100 |
| 200 ppm Triton ® X100 | <10 |
| 120 ppm Triton ® CG110 | 86 |
| 200 ppm EcoSurf ® EH 9 | 51 |
| 200 ppm Tween ® 20 | 146 |
| 200 ppm Tween ® 80 | 154 |
| 200 ppm Tergitol ® 15-S-9 | 51 |

^{‡} active substance concentration in final sample mix

1. A method for depletion or removal of endotoxins from nucleic acids comprising
a) Providing a sample comprising said nucleic acids and endotoxins
b) Subjecting the sample of step a) to a chromatographic separation on a membrane or monolith comprising anion exchange groups
whereby the sample is contacted with a non-ionic detergent selected from the group of alkylglycosides and secondary alcohol alkoxylates or mixtures thereof prior or during the chromatographic separation.

2. The method according to claim 1, wherein b) comprises
i) Loading the sample comprising said nucleic acids and endotoxins onto the membrane or monolith comprising anion exchange groups
ii) Washing the membrane or monolith with a wash buffer
iii) Eluting the nucleic acids bound to the membrane or monolith with an elution buffer.

3. The method according to claim 1, wherein the sample is contacted with the non-ionic detergent prior to b).

4. The method according to claim 3, wherein the sample subjected to the chromatographic separation comprises between 0.01% and 10% (w/v) of the non-ionic detergent.

5. The method according to claim 1, wherein the nucleic acids are contacted with the non-ionic detergent by washing the membrane or monolith with a wash buffer comprising a non-ionic detergent.

6. The method according to claim 5, wherein the wash buffer comprising a non-ionic detergent comprises between 0.01% and 10% (w/v) of the non-ionic detergent.

7. The method according to claim 1, wherein the non-ionic detergent is an alkylglycoside.

8. The method according to claim 1, wherein the nucleic acids comprise plasmid DNA.

9. The method according to claim 1, wherein the nucleic acids are contacted with a solution comprising 0.01 to 10% (w/v) of the non-ionic detergent.

10. The method according to claim 1, wherein in b) a membrane is used.

11. The method according to claim 2, wherein ii) comprises two or more wash steps whereby one wash step is done with a wash buffer comprising ethanol.

12. The method according to claim 11, wherein the method provides for nucleic acids which are depleted from endotoxins more effectively than the otherwise same process but using Triton® X100 as the only detergent.

13. The method according to claim 1, further comprising c) detecting residual endotoxin in the nucleic acids resulting from b).

14. The method according to claim 12, wherein the detection in c) is done by LAL assay.

15. The method according to claim 12, wherein the detection in c) is done directly in the eluate of the chromatographic separation according to b), without any further treatment of the eluate.

16. The method according to claim 1, wherein the nucleic acids consist of plasmid DNA.

17. The method according to claim 1, wherein in b) a hydrogel membrane is used.
